(19) United States
(12) Patent Application Publication
(10) Pub. No.: US 2026/0240675 A1
(43) Pub. Date: Aug. 20, 2026

(54) WEARABLE ASSISTANCE DEVICE WITH AUTOMATIC CLUTCH MECHANISM AND METHOD OF USE

(71) Applicant: Vanderbilt University, Nashville, TN (US)

(72) Inventors: Matthew YANDELL, Nashville, TN (US); Chad ICE, Nashville, TN (US); Karl ZELIK, Nashville, TN (US)

(21) Appl. No.: 19/161,872

(22) PCT Filed: Mar. 7, 2024
(86) PCT No.: PCT/US2024/018812

§ 371 Date: Sep. 3, 2025

Related U.S. Application Data
(60) Provisional application No. 63/450,577, filed on Mar. 7, 2023.

(51) Int. Cl. A61F 5/02; A61F 5/01; F16D 41/14

(52) U.S. Cl. CPC A61F 5/028; F16D 41/14; A61F 2005/0197

(57) ABSTRACT

A wearable assistance device that comprises an upper-body interface, a lower-body interface, and at least one elastic member that couples the upper-body interface to the lower-body interface and is configured to provide an assistive force to the back of the wearer. An automatic clutch mechanism is coupled to the elastic member and is configured for selectively adjusting the assistive force provided by the elastic member. The automatic clutch mechanism may include cooperating spring-biased engagement features that are configured to self-engage. An actuator is provided for switching the clutch mechanism between engaged and disengaged modes. The spring-biased cooperating engagement features are configured to self-engage upon activation of the actuator such that the clutch mechanism is automatically engaged.

### CROSS-REFERENCE TO RELATED APPLICATION

This application claims the benefit of priority of U.S. Provisional Application Ser. No. 63/450,577 filed on Mar. 7, 2023, and entitled “Clutch Mechanisms with Automatic Engagement or Set Point Variability and Uses of the Same,” the content of which is relied upon and incorporated herein by reference in its entirety.

### GOVERNMENT FUNDING

This invention was made with government support under United States Army Grant No. W911NF2120078. The government has certain rights in the invention.

### BACKGROUND

This disclosure relates generally to wearable assistance devices and systems, such as occupational wearable tools and exosuits, and methods of use.

Exos or exosuits (e.g. rigid exoskeletons and soft exosuits) are wearable devices that assist, support, enable or augment human movement, posture, or physical activity through mechanical interaction with the body. Occupational exos are used to provide physical relief and assistance to workers in demanding jobs and to reduce musculoskeletal injury risks and fatigue. For instance, shoulder exos support the arms during overhead work and back exos assist lifting and bending. However, for users to accept and adopt exos, the devices must also be sufficiently comfortable, practical to use, adjustable and not interfere with other critical job tasks or movements. Commercially available occupational exos do not fully meet the needs of some users.

Clutches are used in some exos to turn on assistance when it is needed and to turn off assistance to enhance freedom of movement during other tasks. For safety, reliability, and a good user experience it is important that clutches have consistent mechanical behavior in the presence of a variety of user inputs in different orders of operation and/or manufacturing variations. If, for example, a user only partially engages a clutch (or actuator) and the clutch does not fully engage as a result, conventional clutches might disengage during use or under load when not intended. This may negatively impact user experience or result in an exo not functioning properly, for example, an exo not providing assistance when a user expects that it will. Also, conventional clutches may disengage even while under load if the user accidentally disengages the clutch. Or, if the springs in a clutch are not appropriately balanced or configured, then it can make deactivating the clutch very difficult or cause the clutching function to fail. In addition, a user may also desire to modify the amount of assistance received from the wearable device, which is provided by commercial exosuits. Therefore, there are a variety of limitations of conventional and simple clutches for use with exos. These limitations can negatively impact the user experience, usability, reliability, or the safety of exos. Clutches with enhanced capabilities, such as the self-engagement capability described herein, can overcome some of these limitations and thus improve the functionality of and user experience with an exo.

A need exists for clutches with enhanced capabilities, such as the self-engagement capability described herein, which can overcome these limitations and thus improve the functionality of and user experience with an exo.

### SUMMARY

The present disclosure provides a wearable assistance device that comprises an upper-body interface, a lower-body interface, and at least one elastic member that couples the upper-body interface to the lower-body interface and is configured to provide an assistive force to the back of the wearer. An automatic clutch mechanism is coupled to the elastic member and is configured for selectively adjusting the assistive force provided by the elastic member. The automatic clutch mechanism may include cooperating spring-biased engagement features that are configured to self-engage. An actuator is provided for switching the clutch mechanism between engaged and disengaged modes. The spring-biased cooperating engagement features are configured to self-engage upon movement or activation of the actuator such that the clutch mechanism is automatically engaged when a force is applied to the clutch mechanism via at least one elastic member.

In an embodiment, In an embodiment, the spring-biased cooperating engagement features are configured to remain engaged upon deactivation of the actuator while a force is applied to the clutch mechanism via at least one elastic member. In another embodiment, the actuator is configured to have activated and released positions that correspond to the engaged and disengaged modes, respectively, of the clutch mechanism. In yet another embodiment, the clutch mechanism is configured to be in the engaged mode when the actuator is moved to a released position.

In certain embodiments, the spring-biased cooperating engagement features comprise a rotational pawl and a rotational sprocket in which the rotational pawl engages teeth of the rotational sprocket; the clutch mechanism includes a cable member coupled to the rotational sprocket and coupled to the at least one elastic member, the cable member being configured to reel in and out of the clutch mechanism upon rotation of the rotational sprocket to adjust the tension in the at least one elastic member; and/or the spring of the rotational sprocket is a rotary spring configured to reel in the cable member when the clutch mechanism is disengaged.

In other embodiments, the actuator includes a spring which creates a torque on the clutch mechanism that counters torque created by the spring-biased engagement features on the clutch mechanism such that the clutch mechanism remains at least partially engaged until the assistive force is applied to the clutch mechanism; the actuator includes a transmission coupled to the rotational pawl and coupled to the spring of the actuator; the rotational pawl includes a torsional spring, the rotational sprocket includes a rotary spring, and the spring of the actuator is a linear compression spring; and/or the clutch mechanism is a variable setpoint mechanism configured to adjust the tension in the at least one elastic member.

In some embodiments, the actuator includes a spring which creates a torque on the clutch mechanism that counters torque created by the spring-biased engagement features on the clutch mechanism such that the clutch mechanism remains at least partially engaged until the assistive force is applied to the clutch mechanism; the actuator includes a transmission coupled to the rotational pawl and coupled to the spring of the actuator; the rotational pawl includes a torsional spring, the rotational sprocket includes a rotary spring, and the spring of the actuator is a linear compression spring; and/or the clutch mechanism is a variable setpoint mechanism configured to adjust the tension in the at least one elastic member.

The present disclosure may also provide a method of using an automatic clutch mechanism of a wearable assistance device in which the automatic clutch mechanism is coupled to at least one elastic member of the wearable assistance device that is configured to provide an assistive force to the back of the wearer and the clutch mechanism is configured for selectively adjusting the assistive force provided by the at least one elastic member. The method can comprise the steps of activating an actuator to rotate a first spring-biased engagement feature of the clutch mechanism in a first direction into at least partial engagement with a cooperating second spring-biased engagement feature of the clutch mechanism; and rotating the second engagement feature in a second direction opposite the first direction to fully engage and lock the first and second engagement features together, thereby automatically engaging the clutch mechanism when a force is applied to the clutch mechanism via at least one elastic member.

In one embodiment, the clutch mechanism remains engaged when the actuator activated whether or not the assistive force is applied to the clutch mechanism by the elastic member. In another embodiment, the clutch mechanism is configured to be disengaged when the actuator released and the assistive force is not being applied to the clutch mechanism by the elastic member.

In other embodiments, the method further comprises the step of adjusting the tension in the at least one elastic member by changing a set point of a relative position of the at least one elastic member and the clutch member; the set point is changed by rotating the second engagement feature to reel in and reel out a cable member operatively coupled to the second engagement feature and the at least one elastic member; and/or after the set point is changed, activating the actuator to engage the clutch mechanism.

In other embodiments, the first spring-biased engagement feature is a rotational pawl that has a first spring biasing the pawl in a first direction, and the second spring-biased engagement feature is a rotational sprocket that has a second spring that biases the sprocket in a second direction opposite the first direction; the first spring is a torsional spring and second spring is a rotary spring; the actuator includes a third spring that is a linear compression spring; the actuator creates a torque on the clutch mechanism that counters torque created by the first and second spring-biased engagement features on the clutch mechanism such that the clutch mechanism remains at least partially engaged until the assistive force is applied to clutch mechanism; the actuator temporarily stays in the activated position only as long as force is applied to the actuator and once the force on the actuator is removed, the actuator is released; the clutch mechanism remains in the disengaged mode when the actuator is activated and the clutch mechanism is in the engaged mode when the actuator is released; the actuator temporarily stays in the activated position only as long as force is applied to the actuator and once the force on the actuator is removed, the actuator is released.

In further embodiments, the actuator has first, second, and third positions in which the clutch mechanism self-engages in a first direction when the actuator is in the first position, the clutch mechanism self-engages in a second direction when the actuator is in the second position, and the clutch mechanism is disengaged when the actuator is in the third position, wherein the actuator is released in the third position; the actuator temporarily stays in the first position only as long as force is applied to the actuator in a first direction and once the force on the actuator is removed, the actuator is released; and/or the actuator temporarily stays in the second position only as long as force is applied to the actuator in a second direction and once the force on the actuator is removed, the actuator is released.

In certain embodiments, the clutch mechanism remains engaged when the actuator transitions from the first position to the second position and the assistive force is applied to the clutch mechanism by the elastic member; the clutch mechanism is configured to be disengaged when the actuator is in the second position and the assistive force is not being applied to the clutch mechanism by the elastic member; and/or the method further comprises the step of adjusting the tension in the at least one elastic member by changing a set point of a relative position of the at least one elastic member and the clutch member before engaging the clutch mechanism.

In other embodiments, the set point is changed by rotating the second engagement feature to reel in and reel out a cable member operatively coupled to the second engagement feature and the at least one elastic member; after the set point is changed, activating the actuator to engage the clutch mechanism; the first spring-biased engagement feature is a rotational pawl that has a first spring biasing the pawl in a first direction, and the second spring-biased engagement feature is a rotational sprocket that has a second spring that biases the sprocket in a second direction opposite the first direction; the first spring is a torsional spring and second spring is a rotary spring; the actuator includes a third spring that is a linear compression spring; and/or wherein the actuator creates a torque on the rotational pawl that counters torque created by the first and second spring-biased engagement features on the clutch mechanism such that the clutch mechanism remains at least partially engaged until the assistive force is applied to clutch mechanism.

In another embodiment, the actuator's behavior may be reversed, such that the clutch mechanism is configured to remain in the disengaged mode when the actuator is in the first position. The clutch mechanism is configured to be in the engaged mode when the actuator is in the second position.

In another embodiment, the actuator can have three positions, D, E, and F or release (also referred to herein as “first position”, “second position”, and “third position”). When force is applied to the actuator, it moves between the three states, similar to a rocker light switch or the selector switch on a two-way ratchet wrench. In this embodiment the clutch mechanism self-engages in the clockwise direction when the actuator is in the D position. The clutch mechanism self-engages in the counter-clockwise position when the actuator is in the E position, and the clutch mechanism is disengaged when the actuator is in the F position.

In another embodiment, the actuator temporarily stays in the first or activated position only as long as force is applied to the actuator; once the force on the actuator is removed, a spring forces the actuator returns to the second position.

In another embodiment, the actuator momentarily stays in the second or released position only as long as force is applied to the actuator; once the force on the actuator is removed, a spring forces the actuator returns to the first position.

In another embodiment, the actuator momentarily stays in the D position only as long as force is applied to the actuator in one direction. Once the force on the actuator is removed, the actuator returns to the F position. Additionally, the actuator temporarily stays in the E position only as long as force is applied to the actuator in the opposite direction and once the force on the actuator is removed, the actuator returns to the F position.

This summary is not intended to identify essential features of the claimed subject matter, nor is it intended for use in determining the scope of the claimed subject matter. It is to be understood that both the foregoing general description and the following detailed description are exemplary and are intended to provide an overview or framework to understand the nature and character of the disclosure.

### BRIEF DESCRIPTION OF THE FIGURES

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings, in which:

FIG. 1 is an elevational view of an exemplary wearable assistance device according to the present disclosure, showing a clutch mechanism of the device (shown without a housing cover for clarity);

FIG. 2 illustrates the wearable assistance device of the present disclosure as worn by a user;

FIG. 3 is an exploded view of the clutch mechanism of FIG. 1, showing an actuator of the clutch mechanism and an elastic member of the wearable assistance device coupled to the clutch mechanism;

FIG. 4 illustrates the clutch mechanism of FIG. 1, showing vectors to represent the torque vectors on the clutch system;

FIG. 5 is a view of the clutch mechanism of FIG. 1, showing the clutch mechanism in a disengaged state (State A), partially engaged state (State B), and fully engaged (system locked) state (State C);

FIG. 6 is a view of the clutch mechanism of FIG. 1, showing the clutch mechanism in an actuated disengaged state (State D), in which an actuator of the clutch mechanism has been actuated but the clutch mechanism is not fully engaged, for example when a rotational pawl and rotational sprocket of the clutch mechanism are in contact;

FIG. 7 is a view of another clutch mechanism according to an alternative embodiment of the present disclosure; and

FIG. 8 is an elevational view of another clutch mechanism according to yet another alternative embodiment of the present disclosure.

### DETAILED DESCRIPTION

It is to be understood that the figures and descriptions of the present disclosure may have been simplified to illustrate elements that are relevant for a clear understanding of the present disclosure, while eliminating, for purposes of clarity, other elements found in a typical wearable assistance device or typical method of using a wearable assistance device. Those of ordinary skill in the art will recognize that other elements may be desirable and/or required in order to implement the present disclosure. However, because such elements are well known in the art, and because they do not facilitate a better understanding of the present disclosure, a discussion of such elements is not provided herein. It is also to be understood that the drawings included herewith only provide diagrammatic representations of the presently preferred structures of the present disclosure and that structures falling within the scope of the present disclosure may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting. It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

The present disclosure relates to a wearable assistance device and system, such as an exosuit (also referred to herein as an “exo”), that provides assistive force to a wearer even when the device is only partially engaged (e.g., by partially moving or toggling an actuator, e.g. a switch, of the clutch). Partial engagement might happen, for instance, if a user manually flips a switch, but the switch only rotates part way from one position to another position. The wearable assistance device or system of the present disclosure can also prevent or delay disengagement (e.g. turning off assistance) when the device is under load even if the user changes the actuator from one position to another (e.g., by accidentally or intentionally toggling the switch to its released position). This, for example, can provide convenience to the user because they can toggle the actuator at any time, while also providing safety and reliability of the device by delaying the time at which the clutch physically disengages. The clutch mechanism described in this disclosure also enables a user (also referred to as a “wearer”) to adjust and lock the set point of an associated elastic member to adjust the level of assistance by adjusting the tension in the elastic member. Collectively, those capabilities enable safe, predictable and adjustable mechanical behavior for users.

A wearable assistance device or system 10 of the present disclosure is designed to assist the wearer with physical assistance capabilities, e.g. lifting and bending. The wearable assistance device or system 10 can be an exosuit, such as a back exosuit, that enables users to benefit from physical assistance and musculoskeletal relief.

For purposes of this disclosure, the term “exosuit” refers broadly to a type of exo that is constructed, at least in part, from soft materials such as textiles and elastomers. An exosuit is also known as a soft exoskeleton or soft exo. An exosuit can be powered (e.g., motorized), passive, or quasi-passive. For example, the wearable assistance device of the present disclosure can be a quasi-passive (i.e. using a mode-switching clutch) back exosuit used by wearers conducting logistics or manufacturing tasks who perform strenuous lifting or bending, or used for similar tasks by wearers but worn under a body-worn gear or armor, such as tactical vests by soldiers. The exosuit can include high-tech, breathable fabric, padding, or slip-resistance features in the right places to comfortably conform to the wearer's body.

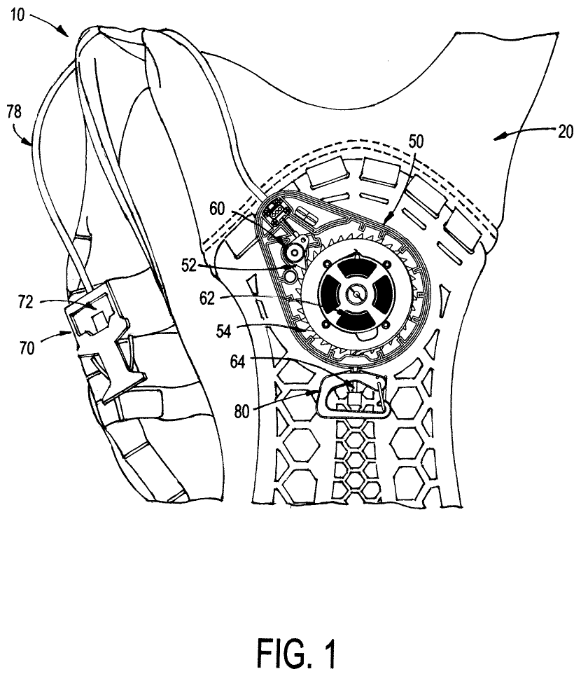

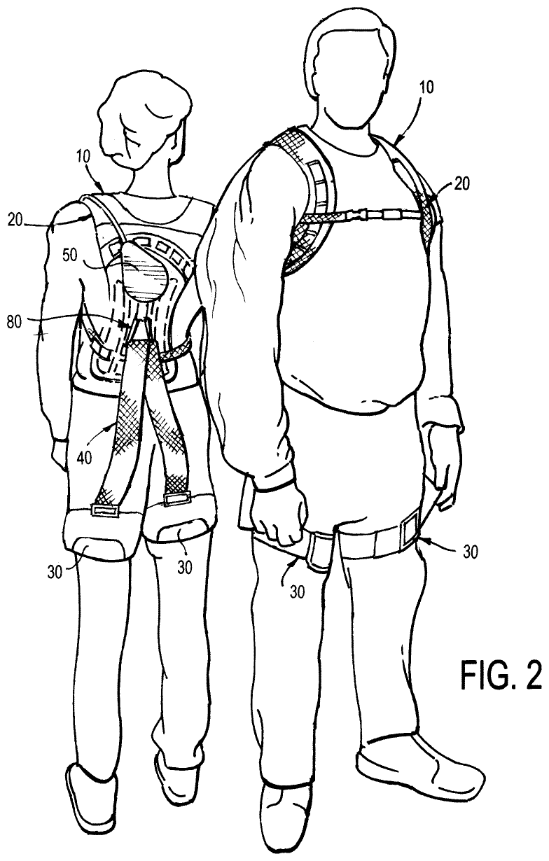

As seen in FIGS. 1 and 2, the wearable assistance device 10 of the present disclosure generally comprises an upper-body interface 20, a lower-body interface 30, one or more elastic members 40 connecting the upper-body and lower-body interfaces 20 and 30, and an automatic clutch mechanism 50 that engages and disengages. The one or more elastic members 40 are configured to provide the assistive force to the back of the wearer, such as when the wear bends or lifts. The clutch mechanism 50 is associated with and operatively coupled to one or more elastic members 40 for selectively adjusting the assistive force provided by the elastic members when the clutch mechanism 50 is disengaged and engaged (also referred to herein as “engaged and disengaged modes”). An actuator 70 (e.g. a switch) associated with the clutch mechanism 50 moves between positions to engage and disengage the clutch mechanism 50. The actuator 70 may be manual or powered (e.g. motorized).

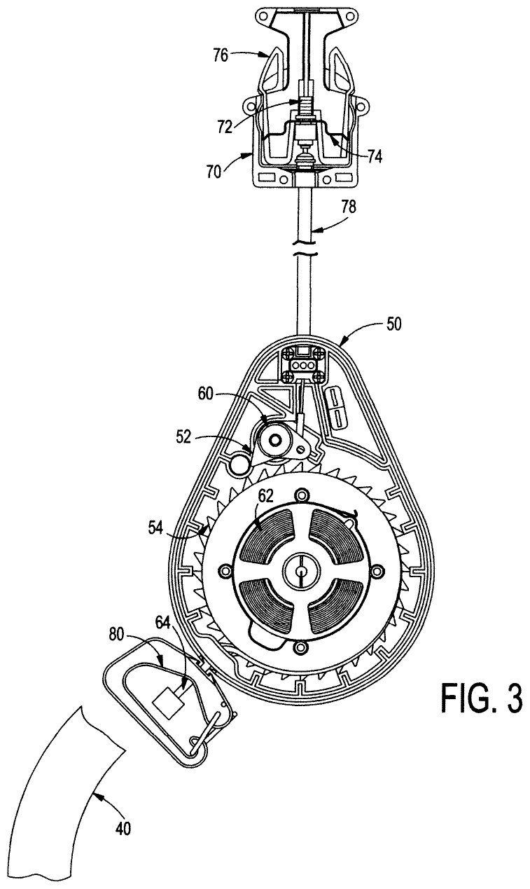

The elastic members 40 are coupled to the automatic clutch mechanism 50 at attachment 80, as seen in FIG. 3. When the clutch mechanism 50 is engaged (i.e. in the engaged mode), elastic members 40 are activated to provide the assistive force to the wearer of the device 10. For instance, when the wearer bends forward or squats down then the elastic members 40 stretch, which generates an assistive torque about the low back which reduces strain on the back and hip muscles and makes lifting easier. In that engaged mode, a load is applied to the clutch mechanism 50 due to the assistive force being applied by the elastic members 40. For safety, the clutch mechanism 50 is configured to remain in its engaged mode when under assistive load even if the actuator 70 is switched to disengage clutch mechanism 50.

The clutch mechanism 50 is designed to ensure consistent mechanical behavior even in the presence of a variety of user inputs in different orders of operation and/or manufacturing variations. For example, if a user were to only partially engages the actuator 70 for the clutch mechanism 50 and clutch mechanism 50 does not fully engage or lock, the clutch mechanism 50 is configured to still automatically engage upon the application of assistance force from elastic member 40. In that situation, the device 10 would complete clutch engagement, that is the clutch mechanism 50 would automatically engage, without needing further user input (e.g. further movement of the actuator 70) to ensure proper behavior of the device 10.

The clutch mechanism 50 will automatically engage after movement or toggling of the actuator 70 to or toward (i.e. partial movement of the actuator) its activated or locked position while an assistive force is being applied to elastic members 40. That can be done by the clutch mechanism 50 via meshing gears, spring-loaded pins, torsion springs or other like mechanisms that can use geometry or stored energy to create motion with some user interaction or without user interaction. The same types of components may also be used to disengage the clutch mechanism 50 automatically after release of the actuator 70.

The term “actuator” refers to the mechanism used to turn assistance on and off. The actuator 70 can be a passive actuator, such as a mechanical switch or button, that the user manually triggers or applies force to toggle modes (e.g. ON and OFF; locked and unlocked; or activated and released). Or the actuator 70 can be a powered actuator, such as an electric motor or solenoid which is controlled by an automated algorithm or from user input or movement. The actuator 70 controls the behavior of the clutch mechanism 50. It can be located on the clutch mechanism 50, or connected to the clutch mechanism 50 via a transmission 78 (e.g., wire, rope, Bowden cable, wireless telemetry), as seen in FIG. 3.

The automatic engagement functionality of the present disclosure can be achieved using cooperating engagement features 52 and 54 that can be spring-biased and configured to be self-engaging. In one embodiment, the cooperating engagement features 52 and 54 include a rotational pawl that cooperates with a rotational sprocket.

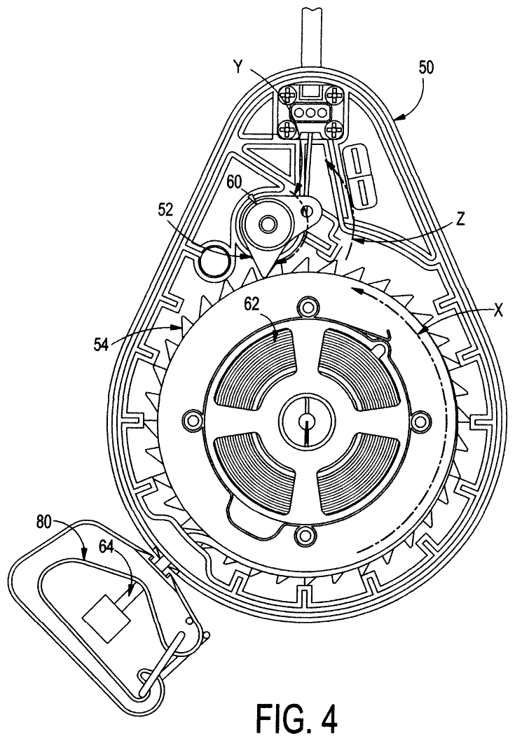

The pawl 52 can be spring-biased in one direction (e.g. clockwise as seen in FIG. 4) via a spring 60 (e.g. torsional) that is nested in a rotational pawl 52; and the sprocket 54 can be spring-biased in the opposite direction (e.g. counter-clockwise as seen in FIG. 4) via a spring 62 (e.g. rotor or rotary spring) that is coupled with the sprocket 54. The spring 62 can be wrapped around or inside a portion of the sprocket 54, for example. FIG. 4 shows the torque vectors (identified as “torque X,” torque Y”, and “torque Z”) on the clutch mechanism 50 from the rotary pawl 52 and associated torsion spring 60, the sprocket 54 and associated rotary spring 62, and the actuator 70 and associated linear spring 72.

In one embodiment, the actuator 70 can be a buckle-style switch, as seen in FIG. 3, that changes modes based on manual user input (i.e. activation). The actuator 70 can be engaged (turned ON or locked) by pulling or pushing on a protrusion 74 that forces the buckle into a locked configuration. In that locked configuration, the spring 72 (which can be located inside the actuator's housing, as seen in FIG. 3) is compressed. The actuator 70 can be disengaged (turned OFF or unlocked) by pinching flanges 76 of the buckle. The compressed spring 72 then recoils, which forces the buckle out of its locked configuration.

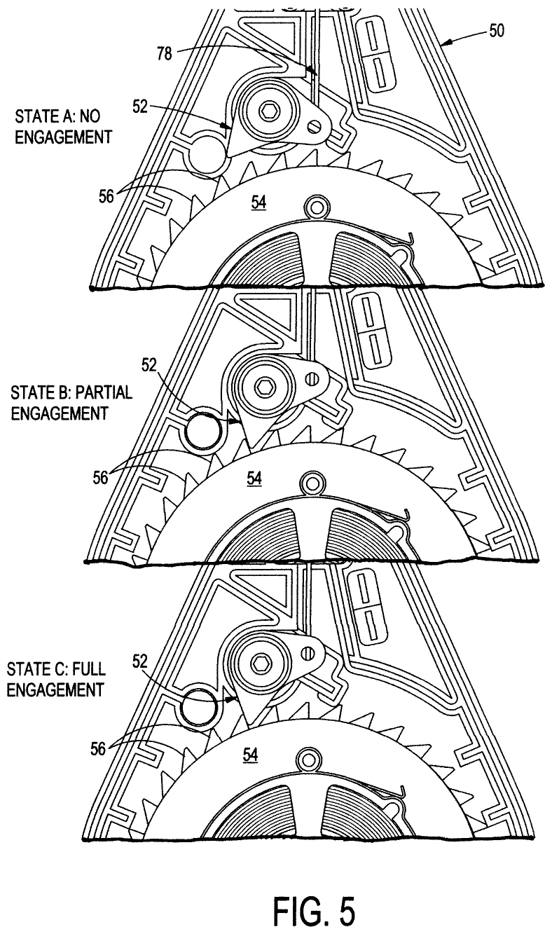

FIG. 5 shows the clutch mechanism 50 in a disengaged state (State A), partially engaged state (State B), and fully engaged (system locked) state (State C). As seen in FIG. 5, the automatic engagement functionality can be achieved when the user activates the actuator 70, such as by pulling the actuator 70 (State A), applying a rotational torque Z on the rotational pawl 52 to rotate against the bias of the spring 60 (e.g. in a counter-clockwise direction in FIGS. 3 and 4). When the clutch mechanism 50 is disengaged (State A), there is minimal or no external load on the clutch mechanism (e.g., minimal or no load from the tension of the elastic member 40 attached to the cable member 64). Next, actuator 70 continues to rotate the pawl 52 until the pawl 52 begins to engage with the teeth 56 of the sprocket 54 (State B). Once the pawl 52 is sufficiently rotated to be in at least partial contact with the sprocket 54 (State B), then continued rotation of the sprocket 54 in the opposite direction (e.g. clockwise in FIGS. 3 and 4) against the bias of the spring 62 will result in an engagement between the pawl 52 and the sprocket 54, e.g. a gear meshing, that automatically forces the clutch mechanism 50 into a fully engaged position (State C). When engaged, the pawl 52 inserts into the teeth 56 of the sprocket 54 which prevents rotation of the sprocket 54 and locks the clutch mechanism 50 in its current orientation.

The continued clockwise rotation of the sprocket 54 (via reversed torque X in FIG. 4) is due to the load on the clutch mechanism 50 as a result of the assistive force being provided by the elastic members 40. In one embodiment, the cable member 64 (cable, rope, or the like) is used with the clutch mechanism 50 and reels in and out thereof, and operatively couples to the elastic bands 40 via an attachment 80. The spring 62 allows the sprocket 54 to reel in the cable member 64 of the clutch mechanism 50 when the clutch mechanism 50 is disengaged (State A) and helps keep the elastic member 40 taut or lightly tensioned, but without restricting or impeding movement of the wearer. The spring 62 and elastic member 40 may be thought of as separate elastic elements acting in series with each other, or as elements of the same elastic member that mechanically couples the upper-body interface to the lower-body interface. This mechanical coupling between the upper-body interface to the lower-body interface may be configured along the back of the wearer, or may couple one or more sides of the upper-body interface to one or more sides of the lower-body interface. There may also be more than two elastic elements that comprise the coupling between the upper-body interface and the lower-body interface. For instance, the cable member 64 may be another element in between the spring 62 and elastic member 40. These elements may be in series or in parallel, or combinations thereof. These elements may be made of any material (e.g. metal, fabric, rubber, webbing), and may be elastic or viscoelastic.

When a user fully engages the actuator 70 and the rotational pawl 52 has a clear opening in the rotational sprocket 54 to complete its rotational arc (as seen in State A, FIG. 5), rotational torque Z will overcome rotational torques X and Y, and the rotational pawl 52 will partially engage (State B, FIG. 5) with the rotational sprocket 54 and the spring 72 in the actuator 70 will partially compress. Upon an assistive force being applied to elastic members 40, torque X will reverse (i.e. to the clockwise direction in FIG. 4) and be applied to the rotational sprocket 54, and the clutch mechanism 50 will fully self-engage (State C, FIG. 5). In addition, the spring 72 in the actuator 70 will partially decompress upon full self-engagement of the clutch mechanism.

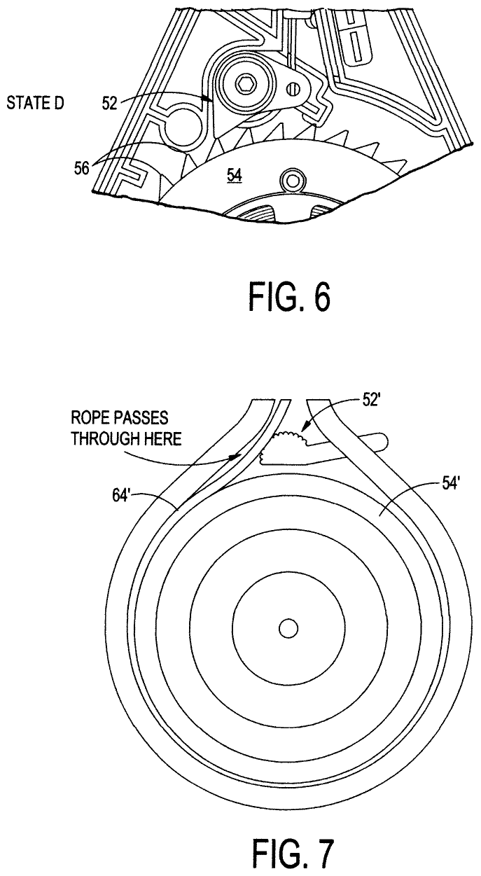

FIG. 6 shows the clutch mechanism 50 in an actuated disengaged state (State D) in which actuator 70 of the clutch mechanism 50 is fully actuated and pawl 52 has rotated (similar to State A), but the clutch mechanism 50 is initially (and temporarily) prevented from fully engaging. In State D, a user engages the actuator 70 for the clutch mechanism 50 but the rotational pawl 52 does not have a clear opening and instead collides with a tooth of the rotational sprocket 54 (State D, FIG. 6). In this situation, the spring 72 in the actuator 70 will still compress to allow the actuator 70 to fully lock, but rotational torque Z will not be able to force the clutch mechanism 50 into the engaged state. However, upon an assistive force being applied to elastic members 40, the resulting torque applied to the rotational sprocket 54 reverses the rotational torque X (i.e. to clockwise in FIG. 4), rotating the rotational sprocket 54 clockwise, thereby providing a clear path for rotational pawl 52 to engage sprocket 54. Rotational torque Z and reversed torque X can then overcome rotational torque Y, and the rotational pawl 52 will partially engage (State B, FIG. 5) with the rotational sprocket 72. Continued application of an assistive force to elastic members 40 will continue the clockwise rotation (i.e. reverse of torque X) of rotational sprocket 54, and the clutch mechanism 50 will fully self-engage (State C, FIG. 5). In addition, the spring 72 in the actuator 70 will partially decompress. Without this device design there could be configurations or singularities of the clutch mechanism where it is not possible (or extremely difficult) to switch actuator modes, which further highlights the usefulness of this design in overcoming these limitations.

If a user only partially engages the actuator 70 for the clutch mechanism 50 and clutch mechanism 50 does not fully engage or lock, the clutch mechanism 50 is configured to still automatically engage if the pawl 52 is sufficiently rotated to be in at least partial contact with the sprocket 54; upon an assistive force being applied to elastic members 40, torque X would reverse to the clockwise direction and will be applied to the rotational sprocket 54, and the clutch mechanism 50 will fully self-engage (State C, FIG. 5). In this situation, the device 10 would complete clutch engagement, that is the clutch mechanism 50 would automatically engage, without needing further user input to the actuator 70 to ensure proper behavior of the device 10. However, if the assistive force is removed without the actuator 70 being completely locked, when the assistive force is removed, clutch mechanism 50 will return to the disengaged state (State A, FIG. 5).

If the actuator 70 is locked and a user disengages the actuator 70 by pressing on the two flanges 76 such that the actuator is unlocked (driving torque Z to zero), the rotational torques X and Y from the rotor spring 62 and torsion spring 60 will force the disengagement of the rotational pawl 52 and the rotational sprocket 54 to full disengagement (State A, FIG. 5) when there is no assistive force being applied to elastic members 40.

When there is an assistive force being applied to elastic members 40, and the actuator 70 is locked and a user disengages the actuator 70 by pressing on the two flanges 76 such that the actuator moves to its unlocked position (driving torque Z to zero), rotational torque X will be reversed (i.e. to the clockwise direction in FIG. 4); torque X will be greater than torque Y and will force the rotational sprocket 52 to remain engaged with the rotational sprocket 54. The clutch mechanism remains in the engaged state (State C, FIG. 5). Upon removal of the assistive force being applied to elastic members 40, rotational torque X reverses to be in the counterclockwise direction, and both rotational torques X and Y from the rotor spring 62 and torsion spring 60 will force the disengagement of the rotational sprocket 52 and the rotational sprocket 54. The clutch mechanism 50 is now disengaged (State A, FIG. 5).

Thus, this clutch design ensures reliable mode-switching into engaged (assistive) mode. Therefore, the user does not need to fully engage the actuator 70 (for example, if the user only partially activates the actuator 70), for the clutch mechanism 50 to be locked (resulting in State C) assuming there is an assistive force applied to elastic members 40. This embodiment could alternatively be achieved with other engagement members (e.g., manual button, rotational knob, electric motor), or with other sprocket or pawl shapes, or directions of movement (e.g., translational rather than linear).

In one embodiment, the clutch mechanism 50 avoids disengaging when it is under high (e.g., ~200 N) load (e.g. load when the assistive force is being provided, where load creates reversed torque X greater than torque Y of torsion spring 60). When the clutch mechanism 50 is fully engaged or locked (State C) and there is an external load on it (e.g., from the tension of the elastic members 40 attached to cable member 64), the user can release the actuator 70 (e.g., switch to the second or released position), but the clutch mechanism 50 will initially remain engaged due to the same dynamics described above that enable the automatic engagement function (resulting in State C). In short, until the external force on the clutch mechanism 50 is reduced, the hold force created by the teeth 56 of the sprocket 54 engaging the pawl 52 maintains the clutch mechanism 50 in the engaged mode. Once the external load is removed (or reduced to a lower force), then the rotational pawl 52 and the sprocket 54 automatically disengage due to the internal spring configuration of the clutch mechanism 50 (State C goes to State B then to State A). Specifically, when there is no external load on the clutch mechanism 50, then releasing the actuator 70 (i.e. switching to the second or release position) causes the rotational pawl 52 to rotate in one direction (clockwise in FIGS. 3 and 4) due to the spring 60 (e.g. torsional spring) nested inside the rotational pawl 52 and causes the sprocket 54 to rotate in the opposite direction (e.g. counter-clockwise in FIGS. 3 and 4) due to the spring 62 (e.g. a rotary spring) operatively connected to the sprocket 54, thereby disengaging the clutch mechanism 50. No additional action is required of the user for this disengagement to occur once forces are low (e.g. ~10 N). And the user can fully toggle the actuator 70 (e.g. switch or button, or other engagement mechanism) into its released position (State A) even when the clutch mechanism 50 is under high load. Thus, this functionality provides both a safety feature in terms of preventing clutch disengagement under assistive force and improving ease of use by allowing the user to toggle (mode switch) the actuator at any time.

The clutch mechanism 50 can be a variable setpoint mechanism to adjust the tension in the elastic member 40 which is operatively connected to the clutch mechanism. The clutch mechanism 50 allows the user to modify the set point of the elastic member attached to the clutch mechanism to change the amount of assistance received from the wearable device, or to change the amount of slack length in the elastic member. The “set point” means the relative position of the clutch mechanism 50 and the elastic member 40, or alternatively the relative position of the clutch mechanism 50 and the attachment 80. In the embodiment shown in FIG. 3, changing the set point changes how much cable 64 is spooled around the sprocket 54. In this circumstance, the clutch mechanism 50 is designed to allow the user to change the set point to any configuration desired by the user in order to promote maximum utility for the user, and the clutch mechanism 50 can engage and disengage at each set point. In particular, the cable member 64 can be wrapped around a portion of the sprocket 54 (such as a spool portion) of the clutch mechanism 50 and can also attach to the elastic member 40, such that when the sprocket 54 is rotated, the cable member 64 can be reeled out and in, to allow for adjustment of the tension in the elastic member 40. Cable member 64 can be reeled out when the user applies an external load which then rotates the sprocket 54 (e.g. clockwise in FIG. 4). The cable member 54 can be reeled in by the action of the bias of the rotary spring 62 to rotate the sprocket 54 in the other direction (e.g. counter-clockwise in FIG. 4) as the user reduces the external load, while the clutch mechanism 50 is in the disengaged mode (State A; FIG. 5). When a desired position of the cable member 64 (which relates to the wearer's desired assistance level from elastic member 40) is reached, the user can then activate the actuator 70 to lock the clutch mechanism (State C; FIG. 5) in that configuration at that desired position. The user can also disengage the clutch mechanism 50 by releasing the actuator 70, adjust the tension in the elastic member 40 or rotation of the sprocket 54 in the same manner as described above, and then re-activate the actuator 70 to lock the clutch mechanism 50 (State C).

The clutch mechanism 50 thus allows adjustment of the set point of the attached elastic member 40 by altering the relative position of the clutch mechanism 50 and the elastic member 40 and activating the actuator 70 at the desired position to lock the clutch mechanism 50. That change in set point can be altered and fully adjusted by the user, by disengaging the clutch mechanism 50, adjusting the set point, then engaging the clutch mechanism 50 again. Those components in the clutch mechanism 50 and/or the actuator 70 can be used in combination with user movement to allow the user to lengthen or shorten the relative position of the clutch mechanism 50 and the elastic member 40 to adjust that set point. The desired clutching function may also be achieved by an adjustable friction clutch, drum brake, latch, buckle, or other clutch mechanism. The lengthening or shortening of the relative position between the clutch 50 and elastic member 40 may also be achieved by an additional actuating mechanism (e.g., electric motor), for instance, that controls the rotation or position of the sprocket 54, spring 62, or elastic member 40.

In the disengaged mode, a load can be applied to the cable member 64 (via elastic member 40) which then rotates the sprocket 54 (e.g. clockwise in FIGS. 3 and 4), which deforms the sprocket's rotary spring 62, thereby increasing the distance between the clutch mechanism 50 and the elastic member 40. This can occur, for instance, when the wearer flexes their trunk or hip. The spring 62 can deform under low force, meaning it does not significantly impede or restrict the wearer's movements, and it can act like a keychain retractor to keep the cable 64 lightly taut and retract in any excess length. The user can then activate the actuator 70, applying torque Z that overcomes torque X and Y, winding up the pawl's torsion spring 60 in a counterclockwise direction, and rotating the pawl 52 counterclockwise and sprocket 54 clockwise, while simultaneously winding up the actuator's spring 72 to put the clutch mechanism 50 in a locked state (State C). The inclusion of the actuator's spring 72 creates a counter-clockwise torque Z on the pawl 52 while in State B and the actuator 70 is locked, which balances the clutch system such that the clutch mechanism 50 remains partially engaged (State B) until an external load or assistive force is applied to the cable member 64. The actuator's spring 72 can be chosen such that the actuator 70 is not too difficult for a user to actuate with one hand, while simultaneously creating enough torque Z to counteract torque X and torque Y regardless of the rotational state of the sprocket 54.

In some embodiments, the clutch mechanism 50 will remain engaged after release of actuator 70 until the external load on the clutch mechanism is low (i.e. drops below a given force threshold) to prevent sudden disengagement of the system (exo) at high force. That may be achieved through geometry (e.g., the external load on the clutch mechanism prevents meshed gears from unlocking), friction (e.g., the external load keeps a friction disk clutch engaged) or other types of engagement. When the external load on the clutch mechanism is reduced, the clutch system may automatically disengage through spring-loaded pins, torsion springs or other mechanisms that can use geometry or stored energy to create motion with some user interaction or without user interaction.

In some embodiments, the pawl 52 can be a different shape (e.g., pin or ball bearing), and it can move differently (e.g. counter-clockwise or translate); the sprocket teeth 56 can be different shapes or the sprocket 54 can rotate clockwise; and/or the spring elements can be of different types (e.g., power, rotor, compression, extension, torsion), or are replaced with electric motors or other powered mechanisms. The actuator's spring 72 can be located in the actuator 70, as seen in FIG. 3. The actuator 70 can be separated from the clutch mechanism 50, and for example, connected thereto via a transmission 78 (e.g. Bowden cable). Alternatively, the springs 60, 62, and 72 can be co-located with the pawl 52 and the sprocket 54 inside of the housing of the clutch mechanism 50.

In an alternative embodiment, the engagement feature of the clutch mechanism is a toothed pawl 52 that can grip a fibrous rope or cable 64 wrapped around an axle 54, as seen in FIG. 6, rather than engaging with a sprocket directly. The toothed pawl releases the rope to disengage. The mechanism may contain various types of elastic elements including rotary springs, torsion springs, and coil springs to achieve the intended effect.

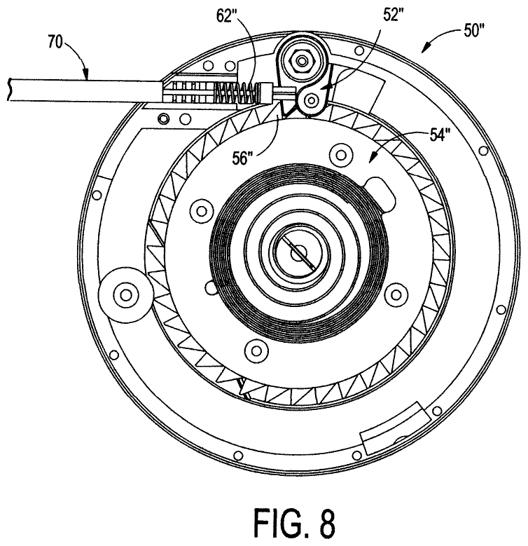

FIG. 8 shows clutch mechanism 50″ according to an alternative embodiment of the present disclosure. In this embodiment, a rotational pawl 52″′ of clutch mechanism 50″ is initially engaged with teeth 56″ of rotational sprocket 54.″ A spring 62″ forces pawl 52″ into engagement with sprocket 54″ when actuator 70 is off and clutch mechanism 50″ is engaged. When actuator 70 is activated, rotational pawl 52″ is pulled out of engagement with the teeth 56″ of sprocket 54″ while compressing spring 62″, thereby disengaging clutch mechanism 50″.

In another embodiment, the actuator can have three states, D, E, and F. When force is applied to the actuator, it moves between the three states, similar to a rocker light switch or the selector switch on a two-way ratchet wrench. In this embodiment, the clutch mechanism 50 self-engages to a locked configuration in the clockwise direction when the actuator is in the D position but can rotate freely in the other direction. Clutch mechanism 50 self-engages to a locked configuration in the counter-clockwise direction when the actuator is in the E position but can rotate freely in the other direction. And the clutch mechanism 50 is disengaged when the actuator is in the F or released position.

In another embodiment, the actuator temporarily stays in the first or activated position only as long as force is applied to the actuator. Once the force on the actuator is removed, a spring forces the actuator to return to the second or released position.

In another embodiment, the actuator temporarily stays in the second position only as long as force is applied to the actuator. Once the force on the actuator is removed, a spring forces the actuator returns to the first or activated position.

In another embodiment, the actuator can have three states, D, E, and F. The actuator momentarily stays in the D position only as long as force is applied to the actuator in one direction (e.g., up); once the force on the actuator is removed, the actuator returns to the F position; additionally, the actuator momentarily stays in the E position only as long as force is applied to the actuator in the opposite direction (e.g., down); once the force on the actuator is removed, the actuator returns to the F position.

The wearable assistance device may include physical assistance and mode-switching capabilities. The wearable assistance device may for example provide back assistance torque (e.g., 10-50 Nm) during bending and lifting, which has been found to be sufficient to reduce back muscle activity and fatigue. The wearable assistance device can incorporate one-handed mode switching (between engaged and disengaged modes) using a manual switch (or other actuator) to allow for many real-life scenarios in which a user/wearer, such as a workman or soldier, may only have one hand available. Alternatively, the device could be designed to mode switch with two hands or no hands (handsfree), such as by using voice-activation or electronic sensors or microcontrollers to control an actuator that performs the mode-switching.

Additional embodiments of the wearable system can be designed to assist other body joints, such as the neck, shoulder, elbow, foot, ankle, knee, or thigh. Different elastic or viscoelastic elements can be used, such as linear and rotational springs and different materials for the springs (e.g., metal, elastomer, fabric). Different methods of clutching, mode-switching or actuation can also be used.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. Those skilled in the art will appreciate various combinations of examples not specifically described or illustrated herein that are still within the scope of this disclosure. In that respect, it is to be understood that the disclosure is not limited to the specific examples set forth and the examples of the disclosure are intended to be illustrative, not limiting.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

Additionally, where a method described above or a method claim below does not explicitly require an order to be followed by its steps or an order is otherwise not required based on the description or claim language, it is not intended that any particular order be inferred. Likewise, where a method claim below does not explicitly recite a step mentioned in the description above, it should not be assumed that the step is required by the claim.

It is noted that the description and claims may use geometric or relational terms, such as right, left, above, below, upper, lower, top, bottom, linear, arcuate, elongated, parallel, perpendicular, etc. These terms are not intended to limit the disclosure and, in general, are used for convenience to facilitate the description based on the examples shown in the figures. In addition, the geometric or relational terms may not be exact. For instance, walls may not be exactly perpendicular or parallel to one another because of, for example, roughness of surfaces, tolerances allowed in manufacturing, etc., but may still be considered to be perpendicular or parallel.

1. A wearable assistance device, comprising:
an upper-body interface and a lower-body interface;
at least one elastic member coupling the upper-body interface to the lower-body interface, the at least one elastic member being configured to provide an assistive force to the back of the wearer;
an automatic clutch mechanism coupled to the at least one elastic member, the clutch mechanism being configured for selectively adjusting the assistive force provided by the at least one elastic member, the automatic clutch mechanism including cooperating spring-biased engagement features that are configured to self-engage; and
an actuator for switching the clutch mechanism between engaged and disengaged modes,
wherein the spring-biased cooperating engagement features are configured to self-engage upon activation of the actuator such that the clutch mechanism is automatically engaged when a force is applied to the clutch mechanism via at least one elastic member.

2. The device of claim 1, wherein the spring-biased cooperating engagement features are configured to remain engaged upon deactivation of the actuator while a force is applied to the clutch mechanism via at least one elastic member.

3. The device of claim 1, wherein the actuator is configured to have activated and released positions that correspond to the engaged and disengaged modes, respectively, of the clutch mechanism.

4. The device of claim 1, wherein the clutch mechanism is configured to be in the engaged mode when the actuator is moved to a released position.

5. The device of claim 1, wherein the spring-biased cooperating engagement features comprise a rotational pawl and a rotational sprocket in which the rotational pawl engages teeth of the rotational sprocket.

6. The device of claim 5, wherein the clutch mechanism includes a cable member coupled to the rotational sprocket and coupled to the at least one elastic member, the cable member being configured to reel in and out of the clutch mechanism upon rotation of the rotational sprocket to adjust the tension in the at least one elastic member.

7. The device of claim 6, wherein the spring of the rotational sprocket is a rotary spring configured to reel in the cable member when the clutch mechanism is disengaged.

8. The device of claim 1, wherein the actuator includes a spring which creates a torque on the clutch mechanism that counters torque created by the spring-biased engagement features on the clutch mechanism such that the clutch mechanism remains at least partially engaged until the assistive force is applied to the clutch mechanism.

9. The device of claim 8, wherein the actuator includes a transmission coupled to a rotational pawl and coupled to the spring of the actuator.

10. The device of claim 8, wherein a rotational pawl includes a torsional spring, a rotational sprocket includes a rotary spring, and the spring of the actuator is a linear compression spring.

11. The device of claim 1, wherein the clutch mechanism is a variable setpoint mechanism configured to adjust the tension in the at least one elastic member.

12. A method of using an automatic clutch mechanism of a wearable assistance device, the automatic clutch mechanism being coupled to at least one elastic member of the wearable assistance device that is configured to provide an assistive force to the back of the wearer and the clutch mechanism being configured for selectively adjusting the assistive force provided by the at least one elastic member, comprising the steps of:
activating an actuator to rotate a first spring-biased engagement feature of the clutch mechanism in a first direction into at least partial engagement with a cooperating second spring-biased engagement feature of the clutch mechanism; and
rotating the second engagement feature in a second direction opposite the first direction to fully engage and lock the first and second engagement features together, thereby automatically engaging the clutch mechanism when a force is applied to the clutch mechanism via at least one elastic member.

13. The method of claim 12, wherein the clutch mechanism remains engaged when the actuator activated whether or not the assistive force is applied to the clutch mechanism by the elastic member.

14. The method of claim 12, wherein the clutch mechanism is configured to be disengaged when the actuator released and the assistive force is not being applied to the clutch mechanism by the elastic member.

15. The method of claim 12, further comprising the step of adjusting the tension in the at least one elastic member by changing a set point of a relative position of the at least one elastic member and the clutch member.

16. The method of claim 15, wherein the set point is changed by rotating the second engagement feature to reel in and reel out a cable member operatively coupled to the second engagement feature and the at least one elastic member.

17. The method of claim 16, wherein after the set point is changed, activating the actuator to engage the clutch mechanism.

18. The method of claim 12, wherein the first spring-biased engagement feature is a rotational pawl that has a first spring biasing the pawl in a first direction, and the second spring-biased engagement feature is a rotational sprocket that has a second spring that biases the sprocket in a second direction opposite the first direction.

19. The method of claim 18, wherein the first spring is a torsional spring and second spring is a rotary spring.

20. The method of claim 19, wherein the actuator includes a third spring that is a linear compression spring.

21. The method of claim 12, wherein the actuator creates a torque on the clutch mechanism that counters torque created by the first and second spring-biased engagement features on the clutch mechanism such that the clutch mechanism remains at least partially engaged until the assistive force is applied to clutch mechanism.

22. The method of claim 12, wherein the actuator temporarily stays in the activated position only as long as force is applied to the actuator and once the force on the actuator is removed, the actuator is released.

23. The method of claim 12, wherein the clutch mechanism remains in the disengaged mode when the actuator is activated and the clutch mechanism is in the engaged mode when the actuator is released.

24. The method of claim 23, wherein the actuator temporarily stays in the activated position only as long as force is applied to the actuator and once the force on the actuator is removed, the actuator is released.

25. The method of claim 12, wherein the actuator has first, second, and third positions in which the clutch mechanism self-engages in a first direction when the actuator is in the first position, the clutch mechanism self-engages in a second direction when the actuator is in the second position, and the clutch mechanism is disengaged when the actuator is in the third position, wherein the actuator is released in the third position.

26. The method of claim 25, wherein
the actuator temporarily stays in the first position only as long as force is applied to the actuator in a first direction and once the force on the actuator is removed, the actuator is released; and
the actuator temporarily stays in the second position only as long as force is applied to the actuator in a second direction and once the force on the actuator is removed, the actuator is released.
